(19) United States
(12) Patent Application Publication
(10) Pub. No.: US 2022/0079935 A1
(43) Pub. Date: Mar. 17, 2022

(54) COMPOUNDS AND TREATMENTS THAT ENHANCE ENTERIC NERVOUS SYSTEM FUNCTION

(71) Applicant: The Johns Hopkins University, Baltimore, MD (US)

(72) Inventors: David Hackam, Baltimore, MD (US); Peng Lu, Lutherville-Timonium, MD (US); Chhinder P. Sodhi, Ellicott City, MD (US)

(21) Appl. No.: 17/421,498

(22) PCT Filed: Jan. 9, 2020
(86) PCT No.: PCT/US2020/012848

§ 371 Date: Jul. 8, 2021

Related U.S. Application Data
(60) Provisional application No. 62/790,184, filed on Jan. 9, 2019.

(51) Int. Cl. A61K 31/4741; A61K 38/18; A61K 35/747; A61K 35/745; A61P 1/04; A61K 31/7008; A61K 45/06

(52) U.S. Cl. CPC A61K 31/4741; A61K 38/185; A61K 35/747; A61K 45/06; A61P 1/04; A61K 31/7008; A61K 35/745

(57) ABSTRACT

The present inventive concepts, compositions and methods are show that the enteric glial serve to restrain the exaggerated TLR4 signaling that occurs in the premature intestinal epithelium via the release of BDNF, and that necrotizing enterocolitis (NEC) develops due to a loss of enteric glia. Compositions and methods of treatment of NEC and related enteric disease in prenatal, premature and neonatal subjects using compositions heretofore unknown for inhibition of NEC or activation of enteric glia are also provided.

### REFERENCE TO RELATED APPLICATIONS

This application claims the benefit of U.S. Provisional Patent Application No. 62/790,184, filed on Jan. 9, 2019, and which is hereby incorporated by reference for all purposes as if fully set forth herein.

### BACKGROUND OF THE INVENTION

Necrotizing enterocolitis (NEC) is the leading cause of death from gastrointestinal disease in premature infants and is characterized by the acute development of intestinal necrosis, with a third of patients dying of their disease. There is no specific treatment for NEC, and overall survival has not changed in the past 40 years. Current thinking suggests that NEC arises from an exuberant inflammatory response to bacterial colonization in the intestine of premature infants, as described in a landmark NICHD consensus conference report and summarized in our recent review in Nature Reviews. Importantly however, the pathways that mediate this heightened pro-inflammatory response, and strategies to reverse it, remain incompletely understood.

Over the past several years, the Hackam lab has discovered that TLR4 signaling in the intestinal mucosa is required for the development of NEC, as mice selectively lacking TLR4 in the intestinal epithelium were protected from NEC compared to wild-type counterparts. We have also determined that TLR4 expression on the intestinal epithelium is higher in the premature human and mouse intestine compared to the full-term intestine, which in mice reflects a novel role played by TLR4 in the regulation of intestinal differentiation. The activation of elevated TLR4 in the postnatal period by colonizing bacteria then results in an increased inflammatory response leading to NEC. Importantly, the factors responsible for the elevated TLR4 signaling, and mechanisms capable of restraining TLR4 in the premature intestine, remain largely unknown.

Therefore, there exists an unmet need to understand the underlying mechanisms for NEC in premature and newborn infants and for compositions and methods of treatment for NEC and related disease.

### SUMMARY OF THE INVENTION

The present inventors now present the novel finding that the enteric glia, which are relatively deficient in the premature bowel, serve to restrain TLR4 signaling in the intestinal epithelium through the release of the peptide brain-derived neurotrophic factor (BDNF). The inventors have also found that a loss of functioning enteric glia in the premature host leads to NEC through unrestrained TLR4 activation. Furthermore, the inventors have now identified a novel class of so-named “glial activating compounds” that induce BDNF release from the enteric glia, and which prevented NEC in mice through reduced TLR4 signaling, and which show the ability to reduce inflammation in intestinal tissue resected from patients with severe NEC.

In accordance with an embodiment, the present invention provides a method for the prevention or treatment of NEC in the intestine of a pre-term or neonatal mammalian subject comprising attenuating enteric glia loss in the intestine of a pre-term or neonatal mammalian subject.

In accordance with an embodiment, the present invention provides a method for the prevention or treatment of NEC in the intestine of a pre-term or neonatal mammalian subject comprising the use of a TLR4 antagonist for reducing TLR4 signaling in the intestine of a pre-term or neonatal mammalian subject.

In accordance with an embodiment, the present invention provides a method for the prevention or treatment of NEC in the intestine of a pre-term or neonatal mammalian subject comprising administering to the subject an effective amount of BDNF.

In accordance with an embodiment, the present invention provides a method for the prevention or treatment of NEC in the intestine of a pre-term or neonatal mammalian subject comprising administering to the subject an effective amount of an anti-oxidant composition.

In accordance with an embodiment, the present invention provides a method for the prevention or treatment of NEC in the intestine of a pre-term or neonatal mammalian subject comprising administering to the subject an effective amount of a glial agonist composition which induces BDNF release in enteric glia.

In accordance with an embodiment, the present invention provides a method for the prevention or treatment of NEC in the intestine of a pre-term or neonatal mammalian subject comprising administering to the subject an effective amount of a composition which prevents enteric glia loss in the intestine of a pre-term or neonatal mammalian subject with necrotizing enterocolitis, as well as the inflammation and dysmotility that characterizes irritable bowel syndrome (IBS) and inflammatory bowel disease (IBD).

### BRIEF DESCRIPTION OF THE DRAWINGS

FIG. 1 illustrates that TLR4 signaling in the intestinal epithelium is required for NEC. 1A) TLR4 SDS-PAGE in control and NEC mice and human ileum. 1B)-1C) Histologic and gross morphology of wild-type TLR4 ^{-/-} or TLR4^{ΔIEC} mice in control and NEC. 1D-E) Human and piglet control and NEC bowel. 1F) IL-1β*** p<0/.001 vs. control. Three separate experiments, each dot=individual subject.

FIG. 2 shows TLR4 in prenatal and postnatal intestine in mouse and human using qRT-PCR. n=5/group P<0.05 vs. e19

FIGS. 3A-3G. Enteric glia restrain TLR4 signaling in the intestinal epithelium and prevent NEC. 3A) Breeding scheme. 3B) qRT-PCR showing increased intestinal TNFα in glia KO mouse reversed by BDNF. 3C) Glial staining with GFAP and PLP1. 3D) Proof of TLR4 reduction in glial-specific TLR4 KO. 3F-G) NEC severity and intestinal TNFα. 5 mice per experiment, *p<0.05, **p<0.01, ***p<0.001 between groups by t-test or ANOVA.

FIGS. 4A-4F. The premature intestine of mice, piglets and human show developmentally low levels of enteric glia, 4A-C) reduced further in NEC by confocal and RT-PCR 4D-F). n=5-9; *p<0.05 by t-test.

FIGS. 5A-5K. BDNF restrains TLR4 signaling in the intestinal epithelium. 5A) enterocytes. 5B) In wild-type mice, BDNF (1 μg/kg) LPS (5 mg/kg) injection for 6 hrs. 5C) qRT-PCR of human ileum freshly harvested from patient with NEC treated with LPS (25 μg/kg) for 6 hr. 5D-H) H&E images. 5I) BDNF ELISA. 5J-K) TNFα and NEC severity. *p<0.05 by ANOVA; 5-10 mice per experiment.

FIGS. 6A-6D. Discovery of novel enteric glia agonist J11. Oral administration of J11 prevents NEC in mice and reduces TLR4 signaling in human tissue. *p<0.05 by t-test or ANOVA. 6 human samples; >6 mice/group.

FIGS. 7A-7B. shRNA mediated knockdown of TrkB in IEC-6 cells (6A) or neonatal mice intestine (6B) prevents BDNF protection of LPS TLR4-induced cytokine expression. *p<0.05 by t-test or ANOVA; >6 mice/group.

FIGS. 8A-8E. In utero injection of fluorescent LPS in fetal stomach (8A, B). Ontogeny of BDNF and TrkB in the intestine (8C-D). (8E) qRT-PCR showing TLR4 knockdown. *p<0.05 by ANOVA or t-test.

FIG. 9. The probiotic bacteria Lactobacillus rhamnosus protects NEC and induces enteric glia accumulation into the lamina propria.

FIG. 10. Representative confocal image of undifferentiated neurosphere from wild-type mice and differentiation into enteric glia.

FIG. 11. Apoptosis (TUNEL) and RT-PCR expression of the pro- and anti-apoptosis genes Bax and Bc12. **P<0.05, 5 mice/group, ANOVA or t-test.

FIGS. 12A-12F. (12A-C) Images stained for the ROS marker dihydroethidium (DHE). (12D-E) Gross and micro images of mouse intestine gavaged nanoparticle. ***p<0.05; >5 mice/group.

FIG. 13. Synthetic analogs of the recently identified TLR4 antagonist.

FIG. 14. Photometric images of TLR4-NFKB luciferase in mice injected with saline or LPS (1 mg/kg) *p<0.05 by t-test; 5 mice/group.

FIGS. 15A-15C. Medicinal chemistry analog preparations for a glial agonist based on a zone model of compound J11.

FIG. 16 is a time response curve showing the BDNF secretion by enteric glial cells stimulated with 10 μM J11. The enteric glial cells (EGC/PK060399egfr, ATCC® CRL-2690™) were treated with 10 μM J11, and the conditional medium were harvested at 2, 6, 24 and 48 hours after treatment. The BDNF secretion was quantified using BDNF ELISA kit following the manufacturer's protocol.

FIG. 17 depicts a time response curve showing the BDNF mRNA expression by enteric glial cells stimulated with 10 μM and 20 μM J11. The enteric glial cells (EGC/PK060399egfr, ATCC® CRL-2690™) were treated with 10 μM and 20 μM J11, and the enteric glial cells were harvested at 2, 6, 24 and 48 hours after treatment.

### DETAILED DESCRIPTION OF THE INVENTION

The present inventive concepts and methods are shown in the first set of studies to identify that the enteric glia serve to restrain the exaggerated TLR4 signaling that occurs in the premature intestinal epithelium via the release of BDNF, and that NEC develops due to a loss of enteric glia.

In some embodiments the present invention provides a novel dendrimer-based nanoparticle compositions linked to an antioxidant (i.e. D-NAC), which when delivered orally, can prevent NEC by attenuating enteric glia loss.

In some embodiments the present invention provides novel a new class of molecules that could prevent or treat human NEC. The lead compound, J11, a 261.23 MW molecule (formula C₁₃H₁₁NO₅) prevents NEC in mice, and reduces inflammation in human NEC tissue ex vivo. These compounds act as glial activators (agonists) and inhibitors in the lumen of the fetal intestine, and can be used assess novel transgenic mouse strains that lack TLR4 on the enteric glia. The present inventive compositions and methods have the potential to directly challenge key concepts in NEC research by showing that the unique susceptibility of the premature infant to NEC occurs, not solely through non-specific impairment in host immunity or barrier function, but (without being held to any particular theory) rather through dysregulated enteric glia resulting in exaggerated TLR4 signaling within the intestinal mucosa, which can now be targeted therapeutically.

In accordance with an embodiment, the present invention provides a method for the prevention or treatment of NEC in the intestine of a pre-term or neonatal mammalian subject comprising attenuating enteric glia loss in the intestine of a pre-term or neonatal mammalian subj ect.

As disclosed herein, the inventors have discovered that that the enteric glia, which are relatively deficient in the premature bowel, serve to restrain TLR4 signaling in the intestinal epithelium through the release of the peptide brain-derived neurotrophic factor (BDNF), and that a loss of functioning enteric glia in the premature host leads to NEC through unrestrained TLR4 activation. As such, administration of compositions which prevent loss or induce growth of enteric glia in the intestine can prevent or treat NEC in the intestine.

In accordance with an embodiment, the present invention provides a method for the prevention or treatment of NEC in the intestine of a pre-term or neonatal mammalian subject comprising administering to the subject a TLR4 antagonist reducing TLR4 signaling in the intestine of a pre-term or neonatal mammalian subject.

As used herein, the term “treat,” as well as words stemming there from, includes preventative as well as disorder remitative treatment. The terms “reduce,” “suppress,” “prevent,” and “inhibit,” as well as words stemming there from, have their commonly understood meaning of lessening or decreasing. These words do not necessarily imply 100% or complete treatment, reduction, suppression, or inhibition.

In accordance with an embodiment, the present invention provides a method for the prevention or treatment of NEC in the intestine of a pre-term or neonatal mammalian subject comprising administering to the subject an effective amount of BDNF.

As used herein, the term “BNDF” means brain derived neurotrophic factor. In, 1982, BDNF, the second member of the “neurotrophic” family of neurotrophic factors, was shown to promote survival of a subpopulation of dorsal root ganglion neurons, and subsequently purified from pig brain. The BDNF gene (in humans mapped to chromosome 11p) has four 5′ exons (exons I-IV) that are associated with distinct promoters, and one 3′ exon (exon V) that encodes the mature BDNF protein. Eight distinct mRNAs are transcribed, with transcripts containing exons I-III expressed predominantly in brain and exon IV found in lung and heart. BDNF shares about 50% amino acid identity with NGF, NT-3 and NT-4/5. Each neurotrophin consists of a noncovalently-1 linked homodimer and contains (1) a signal peptide following the initiation codon; and (2) a pro-region containing an N-linked glycosylation site. Initially produced as proneurotrophins, prohormone convertases such as furin cleave the proneurotrophins (M.W. ˜30kDa) to the mature neurotrophin (M.W. ˜14kDa). Neurotrophins also share a distinctive three-dimensional structure containing two pairs of antiparallel β-strands and cysteine residues in a cystine knot motif.

In some embodiments, the BDNF can be administered orally, and in some embodiments, the BDNF can be administered systemically, such as i.v. or parenterally.

Accordingly, it will be necessary and routine for the practitioner to titer the dosage and modify the route of administration, as required, to obtain the optimal therapeutic effect. A typical daily dosage of BDNF might range from about 0.01 μg/kg to up to about 100 mg/kg or more, preferably from about 0.1 to about 10 μg/kg/day depending on the above-mentioned factors. Typically, the clinician will administer BDNF until a dosage is reached that achieves the desired effect (from 0.01 mg/kg to up to 100 mg/kg or more). The progress of this therapy is easily monitored by conventional assays.

In accordance with an embodiment, the present invention provides a method for the prevention or treatment of NEC in the intestine of a pre-term or neonatal mammalian subject comprising administering to the subject an effective amount of an anti-oxidant and/or anti-inflammatory composition.

In some embodiments, the anti-oxidant and/or anti-inflammatory composition is a dendrimer composition comprising a biologically active agent.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

In accordance an embodiment, the biologically active agent is selected from the group consisting of enzymes, receptor antagonists or agonists, hormones, growth factors, antibodies, oligonucleotides, siRNAs, microRNAs, vitamin A, vitamin C, vitamin E, beta-carotene, and small molecules.

In accordance with another embodiment, the small molecules are selected from the group consisting of anti-inflammatory agents such as steroids, including methyl prednisone, dexamethasone, non-steroidal anti-inflammatory agents, including COX-2 inhibitors, corticosteroid anti-inflammatory agents, gold compound anti-inflammatory agents, immunosuppressive and anti-inflammatory agents, salicylate anti-inflammatory agents, ranibizumab, and minocycline.

“Antioxidant” as used herein, is understood as a molecule capable of slowing or preventing the oxidation of other molecules. Oxidation is a chemical reaction that transfers electrons from a substance to an oxidizing agent. Such reactions can be promoted by or produce superoxide anions or peroxides. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents such as thiols, ascorbic acid or polyphenols. Antioxidants include, but are not limited to, a-tocopherol, ascorbic acid, Mn(III)tetrakis (4-benzoic acid) porphyrin, a-lipoic acid, and n-acetylcysteine.

“Co-administration” as used herein, is understood as administration of one or more agents to a subject such that the agents are present and active in the subject at the same time. Co-adminsitration does not require a preparation of an admixture of the agents or simultaneous administration of the agents.

In accordance with an embodiment, the present invention provides a method for the prevention or treatment of NEC in the intestine of a pre-term or neonatal mammalian subject comprising administering to the subject an effective amount of a composition comprising dendrimer nanoparticles, wherein the dendrimer nanoparticles comprise one or more ethylene diamine-core poly(amidoamine) (PAMAM) hydroxyl-terminated dendrimers covalently linked to at least one biologically active agent, in an amount effective to suppress or inhibit NEC in the intestine of a pre-term or neonatal mammalian subject.

In some embodiments, the dendrimer composition comprises a dendrimer compound conjugated to N-acetyl-cysteine (NAC) which has the structure:

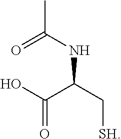

In some embodiments, the dendrimer composition comprises a dendrimer compound conjugated to N-acetyl-L-cysteine amide (NACA), also known as (R)-2-(acetylamino)-3-mercapto-propanamide, N-acetyl-L-cysteinamide, or acetylcysteinamide, has the structure:

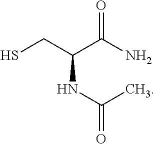

As used herein, the term “PAMAM dendrimer” means poly(amidoamine) dendrimer, which may contain different cores, with amidoamine building blocks. The method for making them is known to those of skill in the art and generally, involves a two-step iterative reaction sequence that produces concentric shells (generations) of dendritic (3-alanine units around a central initiator core. This PAMAM core-shell architecture grows linearly in diameter as a function of added shells (generations). Meanwhile, the surface groups amplify exponentially at each generation according to dendritic-branching mathematics. They are available in generations G0-10 with 5 different core types and 10 functional surface groups. The dendrimer-branched polymer may consist of polyamidoamine (PAMAM), polyester, polyether, polylysine, or polyethylene glycol (PEG), polypeptide dendrimers.

In accordance with some embodiments, the PAMAM dendrimers used can be generation 4 dendrimers, with hydroxyl groups attached to their functional surface groups.

In some embodiments, the dendrimers are in nanoparticle form and are described in detail in International Patent Publication No. WO2009/046446, which is incorporated by reference herein.

It will be understood that the dendrimer compositions used with the methods of the present invention can be in any suitable formulation. Examples of such formulations include one or more of a liposome, a microcapsule, and a nanocapsule.

Embodiments of the invention also include a process for preparing pharmaceutical products comprising the compounds. The term “pharmaceutical product” means a composition suitable for pharmaceutical use (pharmaceutical composition), as defined herein. Pharmaceutical compositions formulated for particular applications comprising the compounds of the present invention are also part of this invention, and are to be considered an embodiment thereof.

In some embodiments, the dendrimer compositions are administered at a concentration of 0.1 mg/kg to 100 mg/kg orally or parenterally, in some embodiments, the concentration administered can be 5 mg/kg, 10 mg/kg, 20 mg/kg, 50 mg/kg, 80 mg/kg and so forth.

In accordance with an embodiment, the present invention provides a method for the prevention or treatment of NEC in the intestine of a pre-term or neonatal mammalian subject comprising administering to the subject an effective amount of a glial agonist.

As used herein, the term “glial agonist” means a compound, composition, peptide or other molecule, which induces BDNF release in enteric glia.

In some embodiments the glial agonist compositions comprise one or more of the following compounds: methscopolamine bromide, ketoprofen, estradiol cypionate, anisodamine hydrobromide, docosanol, oxolinic acid (J11), xylose, benurestat, ioxilan, chlorazanil hydrochloride, OSI-420, laptinib ditosylate, erlotinib HCL, erlotinib, and gefitinib, with or without a pharmaceutically acceptable carrier.

In some embodiments, the glial agonist composition is oxolinic acid (J11). In other embodiments, the composition is selected from analogs of J11 as provided for in the examples herein.

In some embodiments, the glial agonist compositions are administered at a concentration of 0.1 mg/kg to 100 mg/kg orally or peritoneally, in some embodiments, the concentration administered can be 5 mg/kg, 10 mg/kg, 20 mg/kg, 50 mg/kg, 80 mg/kg and so forth.

In accordance with an embodiment, the present invention provides a method for the prevention or treatment of NEC, irritable bowel syndrome (IBS) and inflammatory bowel disease (IBD) in the intestine of a pre-term or neonatal mammalian subject comprising administering to the subject an effective amount of a composition which prevents enteric glia loss in the intestine of a pre-term or neonatal mammalian subject.

In some embodiments, the composition which prevents enteric glia loss is a composition comprising dendrimer nanoparticles wherein the dendrimer nanoparticles comprise one or more ethylene diamine-core poly(amidoamine) (PAMAM) hydroxyl-terminated dendrimers covalently linked to at least one biologically active agent, in an amount effective to suppress or inhibit NEC, irritable bowel syndrome (IBS) and inflammatory bowel disease (IBD) in the intestine of a pre-term or neonatal mammalian subject.

In some embodiments, the dendrimer nanoparticles are conjugated to NAC or NACA.

In some other embodiments, the dendrimer nanoparticles are conjugated to NAC and to additional biologically active agents.

A preferred formulation includes PAMAM dendrimer (4-6 generation) having N-acetyl cysteine bound thereto. In some embodiments, the compositions is NAC conjugated to a fourth generation PAMAM dendrimer (PAMAM-NH₂) using N-succinimidyl 3-(2-pyridyldithio)propionate (SPDP) as a linker, as disclosed in WO2017/074993 and incorporated by reference herein as if set forth in its entirety.

In some embodiments, the composition which prevents enteric glia loss is a probiotic bacterial composition.

In some embodiments, the probiotic composition comprises Lactobacillus rhamnosus. In some other embodiments the probiotic composition comprises Bifidobacterium. In some embodiments, the amount of probiotic composition administered is between about 1×10⁴ to about 1×10⁶ CFU.

In some embodiments, the composition which prevents enteric glia loss is a composition comprising a TLR4 antagonist compound.

As used herein, the term “TLR4 antagonist compound” is any compound, glycoside, peptide, or small molecule, which reversibly or irreversibly inhibits TLR4 binding to LPS or other TLR4 agonists.

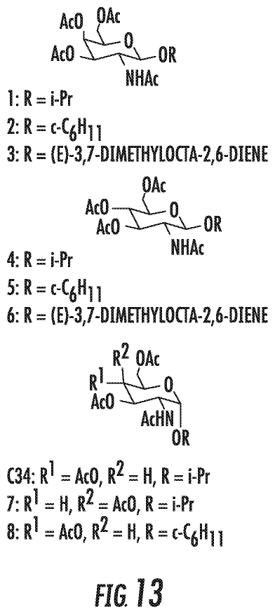

In some embodiments, the TLR4 antagonists of the present invention comprise tetra-acetylated aminoglycosides. In one embodiment, the TLR4 antagonist is a compound identified herein as “C34” and is depicted as compound 1 on FIG. 13. Compounds 2 thru 8, as identified in FIG. 13 are also included in the group of TLR4 antagonists of the present invention.

In accordance with an embodiment, the present invention provides a TLR4 antagonist having the following formula:

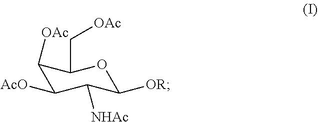

wherein R is H or a straight or branched chain lower alkyl, lower alkenyl, lower alkynyl, alkoxy, cycloalkyl, heterocycloalkyl, aryl, arylalkyl, heteroaryl, and heteroarylalkyl, or a pharmaceutically acceptable salt, solvate, stereoisomer, or derivative thereof

In some embodiments, in the TLR4 antagonist of formula I, R is selected from the group consisting of isopropyl, cyclohexyl, and (E)-3,7,-dimethylocta-2,6-diene.

In some embodiments, in the TLR4 antagonist of formula I is in a composition with a pharmaceutically acceptable carrier.

In some embodiments, in the TLR4 antagonist of formula I is in a composition with at least one additional biologically active agent in a pharmaceutically acceptable carrier.

In accordance with an embodiment, the present invention provides a TLR4 antagonist having the following formula:

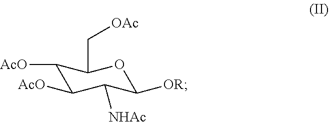

wherein R is H or a straight or branched chain lower alkyl, lower alkenyl, lower alkynyl, alkoxy, cycloalkyl, heterocycloalkyl, aryl, arylalkyl, heteroaryl, and heteroarylalkyl, or a pharmaceutically acceptable salt, solvate, stereoisomer, or derivative thereof.

In some embodiments, in the TLR4 antagonist of formula II, R is selected from the group consisting of isopropyl, cyclohexyl, and (E)-3,7,-dimethylocta-2,6-diene.

In some embodiments, in the TLR4 antagonist of formula II is in a composition with a pharmaceutically acceptable carrier.

In some embodiments, in the TLR4 antagonist of formula II is in a composition with at least one additional biologically active agent in a pharmaceutically acceptable carrier.

In accordance with an embodiment, the present invention provides a TLR4 antagonist having the following formula:

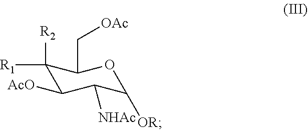

wherein R, Ri, and R₂ are each independently H or a straight or branched chain lower alkyl, lower alkenyl, lower alkynyl, alkoxy, cycloalkyl, heterocycloalkyl, aryl, arylalkyl, heteroaryl, and heteroarylalkyl, or a pharmaceutically acceptable salt, solvate, stereoisomer, or derivative thereof

In some embodiments, in the TLR4 antagonist of formula III, R is isopropyl or cyclohexyl, and R₁ and R₂ are independently H or acetoxy.

In some embodiments, in the TLR4 antagonist of formula II is in a composition with a pharmaceutically acceptable carrier.

In some embodiments, in the TLR4 antagonist of formula II is in a composition with at least one additional biologically active agent in a pharmaceutically acceptable carrier.

The terms “amine” and “amino” are art-recognized and include both unsubstituted and substituted amines. A primary amine carries two hydrogens, a secondary amine, one hydrogen and another substituent and a tertiary amine, the two hydrogens are substituted. The substituents for one or both of the hydrogens can be, for example, and alkyl, an alkenyl, and aryl, a cycloalkyl, a cycloalkenyl, a heterocycle, a polycycle and so on. If both hydrogens are substituted with carbonyls, the carbonyl framed nitrogen forms an imide.

The term “alkylamine” includes an amine group, as defined above, having a substituted or unsubstituted alkyl attached thereto.

The term “amido” is art-recognized as an amino-substituted carbonyl.

The term “alkylthio” is art-recognized and includes and alkyl group, as defined above, having a sulfur radical attached thereto. In certain embodiments, the “alkylthio” moiety is represented by one of —S-alkyl, —S-alkenyl, —S-alkynyl and so on. Representative alkylthio groups include methylthio, ethylthio and the like.

The term “carbonyl” is art-recognized and includes a C=0 structure. Carbonyls are involved in esters; carboxyl groups; formates; thiocarbonyls; thioesters; thiocarboxylic acids; thioformates; ketones; and aldehydes.

The terms “alkoxyl” and “alkoxy” are art-recognized and include an alkyl group, as defined above, having an oxygen radical attached thereto. Representative alkoxyl groups include methoxy, ethoxy, propyloxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl and so on.

The term “sulfonate” is art-recognized and includes a moiety wherein a sulfur atom carries two double bonded oxygens and a single bonded oxygen.

The term “sulfate” is art-recognized and includes a moiety that resembles a sulfonate but includes two single bonded oxygens.

The terms “sulfonamide,” “sulfamoyl,” “sulfonyl,” and “sulfoxido” are art-recognized and each can include a variety of R group substituents as described herein.

The terms phosphoramidite” and “phophonamidite” are art-recognized.

The term “selenoalkyl” is art-recognized and includes an alkyl group having a substituted seleno group attached thereto. Exemplary “selenoethers” which may be substituted on the alkyl are selected from one of —Se-alkyl, —Se-alkenyl, —Se-alkynyl and so on.

Substitutions may be made to alkenyl and alkynyl groups to produce, for example, aminoalkenyls, aminoalkynyls, amidoalkenyls, iminoalkenyls, iminoalkynyls, thioalkenyls, thioalkynyls, carbonyl-substituted alkenyls or alkynyls.

A hydrocarbon is an art recognized term and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer carbon atoms. Likewise cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls,” the latter of which refers to alkyl moieties having substituents replacing hydrogen on one or more carbons of the hydrocarbon backbone. Such substituents may include, for example, a halogen, a hydroxyl, a carbonyl (such as a carboxyl, an alkoxycarbonyl, a formyl or an acyl), a thiocarbonyl (such as a thioester, a thioacetate, or a thioformate), an alkoxyl, a phosphoryl, a phosphonate, a phosphinate, an amino, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN and the like.

The term “aralkyl” is art-recognized, and includes aryl groups (e.g., an aromatic or heteroaromatic group).

The terms “alkenyl” and “alkynyl” are art-recognized, and in an organic molecule, generally includes an atom of any element other than carbon or hydrogen. Illustrative heteroatoms include boron, nitrogen, oxygen, phosphorus, sulfur, and selenium.

The term “aryl” is art-recognized, and includes 5-, 6-, and 7-membered single ring aromatic groups that may include from zero to four heteroatoms, for example, benzene, pyrole, furan, thiophene, imidazole, oxazole, thiazole, triazole, pyrazole, pyridine, pyrazine, pyridazine and pyrimidine, and the like. Thos aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydyl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls, and/or heterocyclyls, or rings joined by non-cyclic moieties.

In some embodiments, the amount of TLR4 antagonist compositions administered is between about of 0.1 mg/kg to 100 mg/kg orally or peritoneally, in some embodiments, the concentration administered can be 5 mg/kg, 10 mg/kg, 20 mg/kg, 50 mg/kg, 80 mg/kg and so forth. In some additional embodiments, the TLR4 antagonists can be administered in conjunction with one or more other compositions described herein, including, for example, D-NAC, oxolinic acid, and other biologically active agents.

In accordance with some embodiments, the present invention provides glial agonist compounds which stimulate the release of BDNF from enteric glia having the following formula:

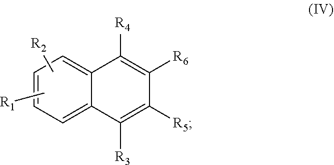

wherein R₁ and R₂ are independently H, C₁-C₆ alkyl, halo, alkoxy, alkyhalo, alkoxyhalo, aryl, heteroaryl, and when R₁ and R₂ are in combination, O(CH₂)ₙO, R₃ is H, C₁-C₆ alkyl, halo, alkoxy, alkyhalo, alkoxyhalo, aryl, heteroaryl, and substituted heteroaryl, R₄ and R₅ are alternatively H or O, R₆ is H, C₁-C₆ alkyl, halo, alkoxy, alkyhalo, alkoxyhalo, sulfonamide, alkysulfamido, aryl, heteroaryl or a pharmaceutically acceptable salt, solvate, stereoisomer, or derivative thereof.

In some embodiments, in the compound of formula IV, R₁ and R₂ are selected from the consisting of H, CH₃, Cl, F, OCH₃, CF₃, OCF₃, OCHF₂ and heteroaryl.

In some embodiments, in the compound of formula IV, R₃ is selected from the consisting of methyl, propyl, butenyl, pentenyl, and CH₂R₇, wherein R₇ is selected from the group consisting of methyl, ethyl, propyl, CH₂CF₃, CH₂OH, and heteroaryl.

In some embodiments, in the compound of formula IV, R₆ is CO₂H, heteroaryl, and SO₂NHR₈ wherein R₈ is H, CH₃, heteroaryl, and phenyl.

In some embodiments, the compound of formula IV is in a composition with a pharmaceutically acceptable carrier.

In some embodiments, the compound of formula IV is in a composition with at least one additional biologically active agent in a pharmaceutically acceptable carrier.

In some embodiments, the present invention provides compounds which stimulate the release of BDNF from enteric glia having the following formula:

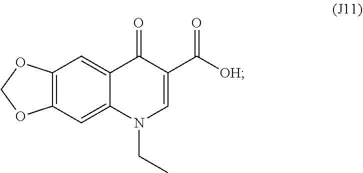 [SMILES: CCn1cc(C(=O)O)c(=O)c2cc3c(cc21)OCO3]

also known as oxolinic acid, or a pharmaceutically acceptable salt, solvate, stereoisomer, or derivative thereof.

In some embodiments, the compound of J11 is in a composition with a pharmaceutically acceptable carrier.

In some embodiments, the compound of J11 is in a composition with at least one additional biologically active agent in a pharmaceutically acceptable carrier.

In accordance with some embodiments, the present invention provides compounds which stimulate the release of BDNF from enteric glia having the following formula:

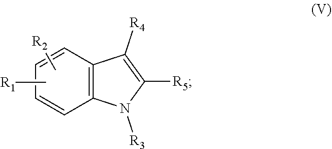

wherein R₁ and R₂ are independently H, C₁-C₆ alkyl, halo, alkoxy, alkyhalo, alkoxyhalo, aryl, heteroaryl, and when R₁ and R₂ are in combination, O(CH₂)ₙO, R₃ is H, C₁-C₆ alkyl, halo, alkoxy, alkyhalo, alkoxyhalo, aryl, heteroaryl, and substituted heteroaryl, R₄ is H, C₁-C₆ alkyl, halo, alkoxy, alkyhalo, alkoxyhalo, sulfonamide, alkysulfamido, aryl, heteroaryl, Rs is alternatively H or O, or a pharmaceutically acceptable salt, solvate, stereoisomer, or derivative thereof.

In some embodiments, in the compound of formula V, R₁ and R₂ are selected from the consisting of H, CH₃, Cl, F, OCH₃, CF₃, OCF₃, OCHF₂ and heteroaryl.

In some embodiments, in the compound of formula V, R₃ is selected from the consisting of methyl, propyl, butenyl, pentenyl, and CH₂R₇, wherein R₇ is selected from the group consisting of methyl, ethyl, propyl, CH₂CF₃, CH₂OH, and heteroaryl.

In some embodiments, in the compound of formula V, R₄ is CO₂H, heteroaryl, and SO₂NHR₈ wherein R₈ is H, CH₃, heteroaryl, and phenyl.

In some embodiments, the compound of formula V is in a composition with a pharmaceutically acceptable carrier.

In some embodiments, the compound of formula V is in a composition with at least one additional biologically active agent in a pharmaceutically acceptable carrier.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

In addition to the compositions and methods above, those compositions can be co-administered or sequentially administered with an antibiotic agent. Examples of antibiotic agents suitable for use in pharmaceutical composition heretofore described above and one or more antibiotic agents include, for example, quinolone antibiotics, such as levofloxacin, ciprofloxacin, ibafloxacin, pradofloxacin, rosoxacin, and sarafloxacin. Other suitable antibiotics are trimethoprim-sulfamethoxazole mixtures such as Bactrim®. Alternatives include rifaximin and azithromycin. Dosages vary with the weight and age of the subject to be treated. Typically, quinolone antibiotics and trimethoprim-sulfamethoxazole mixtures are given at dosages between 250 and 500 mg daily. For trimethoprim-sulfamethoxazole, the dosages are generally between about 5 mg/kg and 25 mg/kg. For rifaximin the dosage ranges from 100 mg to about 500 mg, with 200 mg being preferred. Azithromycin is typically administered at 250-500 mg/day. The dosages required are well within the knowledge of those of ordinary skill in the art.

The term, “peptide,” as used herein, includes a sequence of from four to sixteen amino acid residues in which the a-carboxyl group of one amino acid is joined by an amide bond to the main chain (a- or (3-) amino group of the adjacent amino acid. The peptides provided herein for use in the described and claimed methods and compositions can be cyclic.

The precise effective amount for a premature or neonatal human subject will depend upon the severity of the subject's disease state, general health, age, weight, gender, diet, time and frequency of administration, drug combination(s), reaction sensitivities, and tolerance or response to therapy. Routine experimentation can determine this amount and is within the judgment of the medical professional. Compositions may be administered individually to a patient, or they may be administered in combination with other drugs, hormones, agents, carriers and the like.

With respect to compositions described herein, the carrier can be any of those conventionally used, and is limited only by physico-chemical considerations, such as solubility and lack of reactivity with the active compound(s), and by the route of administration. The carriers described herein, for example, vehicles, adjuvants, excipients, and diluents, are well-known to those skilled in the art and are readily available to the public. It is preferred that the carrier be one which is chemically inert to the active agent(s), and one which has little or no detrimental side effects or toxicity under the conditions of use. Examples of the carriers include soluble carriers, such as known buffers, which can be physiologically acceptable (e.g., phosphate buffer), as well as solid compositions such as solid-state carriers or latex beads.

The carriers or diluents used herein may be solid carriers or diluents for solid formulations, liquid carriers or diluents for liquid formulations, or mixtures thereof

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, or suspensions. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In addition, in some embodiments, the compositions or derivatives thereof of the present invention, may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzlkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular active agent contained in the compositions, as well as by the particular method used to administer the composition. Accordingly, there are a variety of suitable formulations of the pharmaceutical compositions of the invention.

In accordance with an embodiment of the present invention, the present invention provides the use of a medicament for treating a disease in a subject, which can encompass many different formulations known in the pharmaceutical arts, including, for example, intravenous and sustained release formulations. With respect to the inventive methods, the disease can include any gastrointestinal disease, for example, NEC, irritable bowel syndrome (IBS) and inflammatory bowel disease (IBD).

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

### EXAMPLES

In all experiments, extreme care is taken to ensure full data transparency, including the use of scatter plots as opposed to bar graphs, thereby ensuring full accounting of all data points. Care is always taken to ensure mouse experiments are performed at the same age between groups. Although we have not observed an effect of gender, we are vigilant to the possibility that NEC development may be impacted by the gender, and so we randomize to gender in all cases. Studies performed in any cultured cell line (i.e. IEC-6 cells or the glia cell line EGC/PK060399) are be used up to passage 15 to ensure that no spontaneous gene alteration occurs, and all mouse genotypes will be confirmed by RT-PCR prior to experimentation. All experimental details are either described in the application or are fully referenced in our prior publications for reproducibility.

### Example 1

The development of NEC requires TLR4 activation in the premature intestinal epithelium.

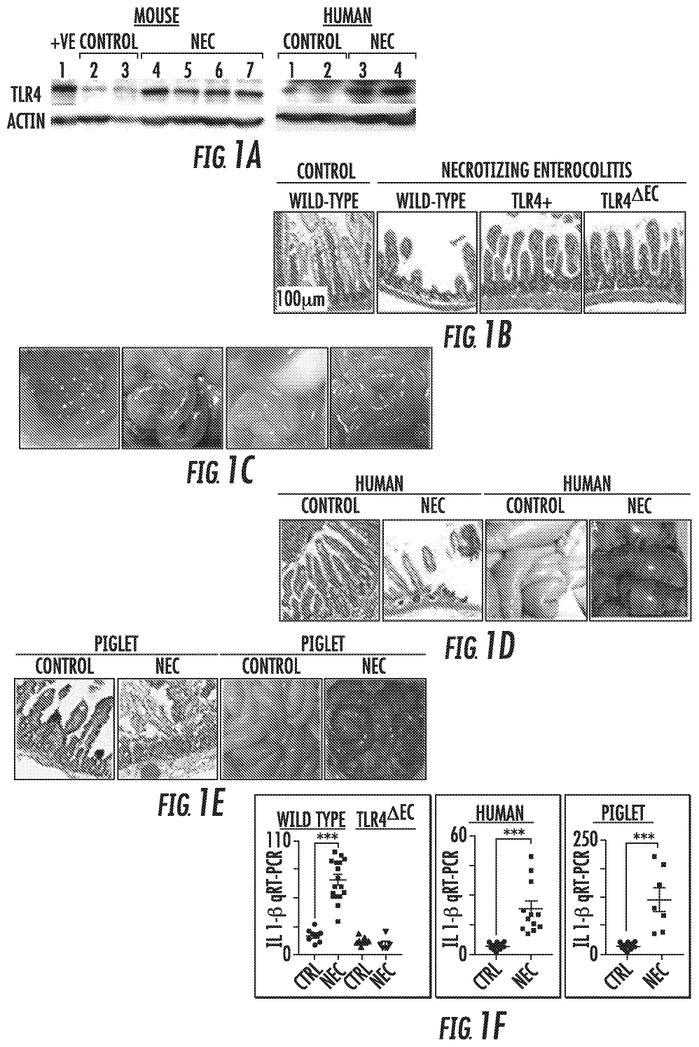

The present inventors have discovered that the development of NEC requires activation of the receptor for lipopolysaccharide (LPS), namely Toll-like receptor 4 (TLR4), in the premature intestinal epithelium by microbes that colonize the gastrointestinal tract, findings which are now supported by many other labs. Specifically, as shown in FIG. 1, mice that lack TLR4 in the intestinal epithelium (herein referred to as TLR4^{ΔIEC} mice) or globally (TLR4^{-.-}), are protected from the development of NEC as compared with wild-type mice, when subjected to our well validated NEC model.

In the inventors' NEC model, 7d old mice are gavaged with a combination of Similac Advance infant formula (Abbott Nutrition):Esbilac canine milk replacer (PetAb) in a 2:1 ratio at 50 μl/g every 3 hours, and subjected to 10 minutes of hypoxia (5% O₂ 95% N₂ in a Billups-Rothenberg chamber) twice daily for four days, and then orally gavaged dysbiotic bacteria (1×10⁵ cfu/ml) obtained from the stool of a patient with severe NEC (see our recent JCI paper for more model details⁷). This protocol induces patchy intestinal necrosis and mucosal disruption which mimics human NEC in wild-type mice, but not in either TLR4^{ΔIEC} or TLR4^{-/-} littermates.

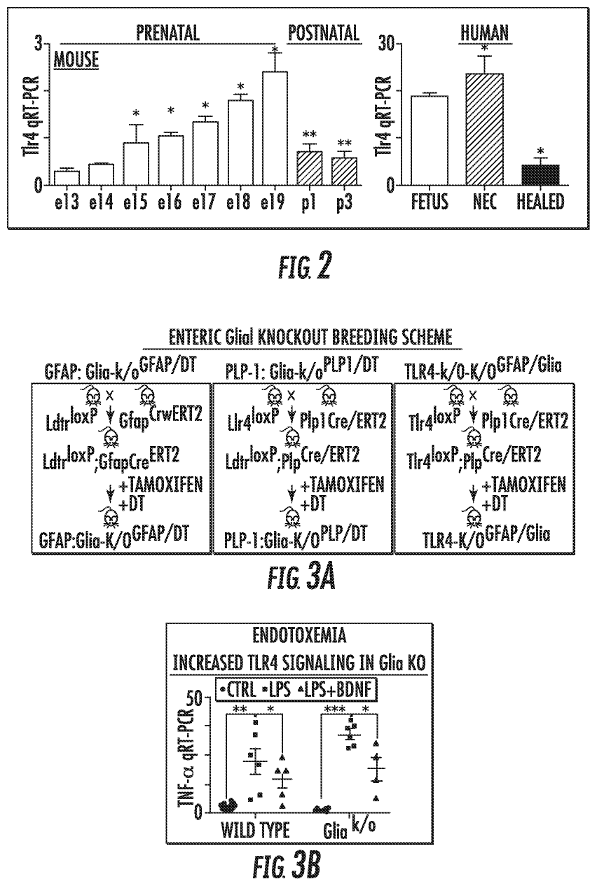

In seeking to understand how TLR4 signaling in the intestinal epithelium leads to NEC, the inventors have also shown that TLR4 activation in the intestinal epithelium leads to apoptosis of the intestinal epithelial cells as well as increased expression of the proinflammatory cytokine IL-1β, which combine to cause mucosal injury (FIG. 1). The subsequent translocation of bacteria across the intestinal epithelium into the mesenteric circulation activates TLR4 on the endothelial lining, leading to mesenteric vasoconstriction and the intestinal ischemia that characterizes NEC. TLR4 expression is higher in the premature as compared with the full-term intestine in mice and humans, and rises in utero as the intestine develops, and then falls shortly after birth, reflecting its role in regulating normal intestinal cell fate specification (FIG. 2). Thus, in a baby born prematurely, TLR4 expression is still elevated in the intestine, and so becomes activated postnatally by colonizing microbes, leading to NEC. In support of the clinical relevance of this model, activating mutations in the TLR4 pathway predispose to NEC in humans, while treatment of mice and human tissue ex vivo with our novel TLR4 antagonist prevents NEC.

### Example 2

The enteric glia restrain TLR4 signaling in the intestinal epithelium and prevent NEC.

Enteric glia are a component of the enteric nervous system (ENS), a complex network of neurons and glia that controls many aspects of bowel function. While enteric neurons govern intestinal motility, enteric glia support the neurons around which they are located, and maintain the intestinal epithelial barrier, given their location in the lamina propria. In a study by Bush et al., mice selectively lacking enteric glia developed spontaneous fulminant jejuno-ileitis that resembled NEC. This result was confirmed by Savidge et al. who showed that glial ablation led to intestinal injury via barrier dysfunction, using a different mouse model. While a recent paper by Rao et al. disputed these earlier findings by attributing the prior results to epithelial effects, none of the reports examined pups in the postnatal period when NEC develops.

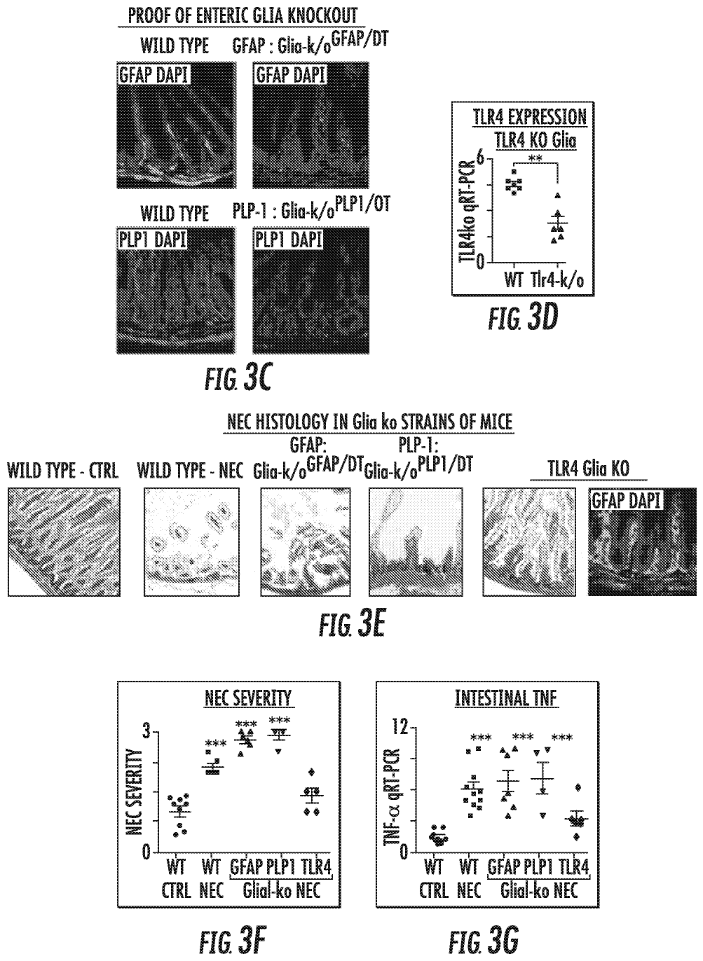

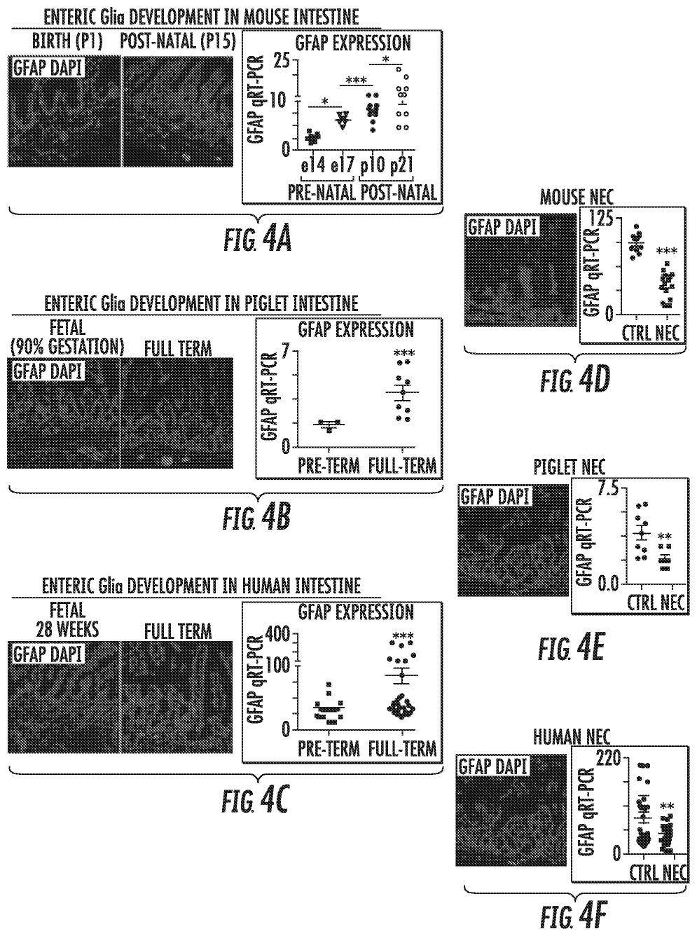

The present inventors have generated two different glial-deficient strains of mice, by breeding ROSA26iDTR with both GFAP-cre/ERT2 and Plp1-cre/ERT2 (see FIG. 3 for breeding scheme and evidence of glial deletion after tamoxifen injection), and determined that the enteric glia restrain TLR4 signaling in the intestinal epithelium, as evidenced by increased cytokine release in the intestinal epithelium of mice lacking enteric glia after injection with the TLR4 ligand LPS (50 μg/ml). It was noted that there is no epithelial expression of GFAP or PLP-1 in the neonatal epithelium (FIG. 3), excluding an epithelial effect, as was attributed in adult mice. Strikingly, enteric glial deficient mice show significantly increased NEC severity compared to wild type mice (FIG. 3). Importantly, the premature intestine of mice, piglets and humans express reduced enteric glia at baseline (FIG. 4) compared with full term, while the development of NEC in mice, piglets and humans reveals a further reduction in the expression of enteric glia as compared to premature bowel without NEC (FIG. 4), linking the lack of enteric glia with the development of NEC. To evaluate whether the loss of glia is a cause, rather than a consequence of NEC, we note that enteric glia express abundant TLR4, and thus generated mice that specifically lack TLR4 on the enteric glia (“glia-TLR4-KO”). As shown in FIG. 3, glia-TLR4-KO mice do not reveal a loss of glia (FIG. 3E) and are significantly protected from NEC as compared with wild type mice (FIG. 3E-G).

### Example 3

The enteric glia-derived peptide BDNF restrains TLR4 signaling in the intestinal epithelium.

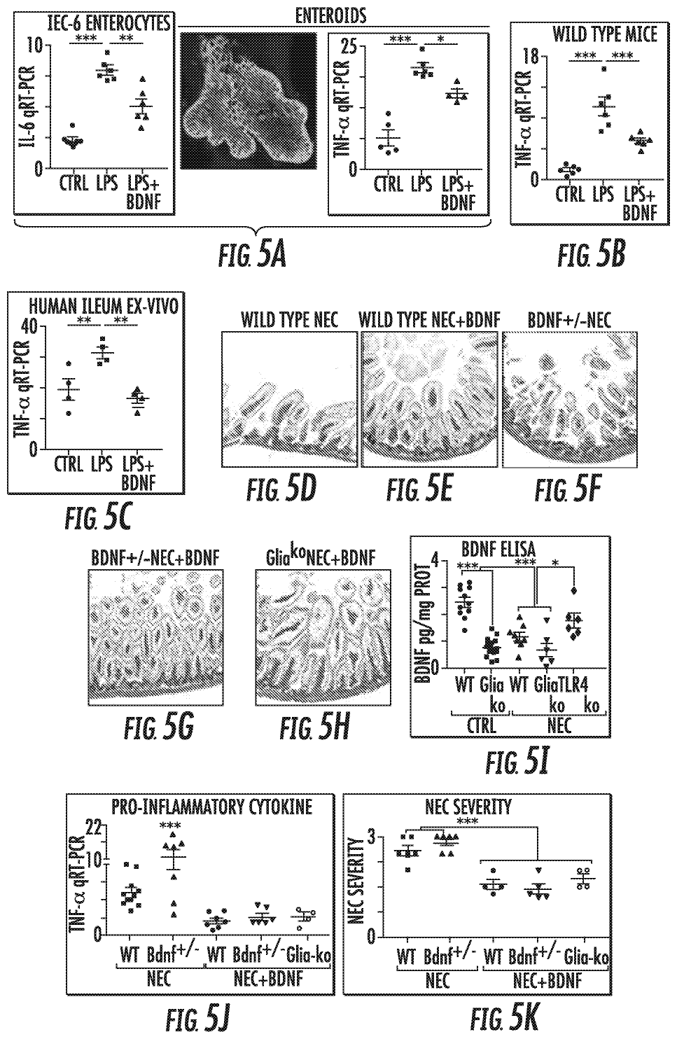

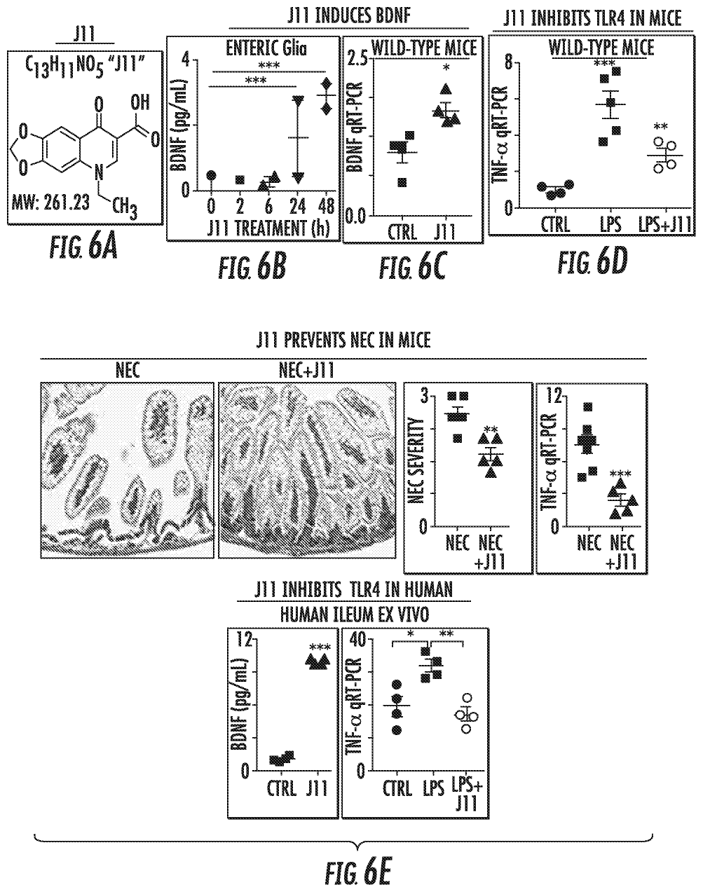

The inventors provide seven lines of evidence which show that the most abundant glial-derived peptide, namely BDNF, which signals through its receptor Tropomyosin receptor kinase B (TrkB), mediates the protective effects of the enteric glia on TLR4 signaling and NEC development. First, treatment of cultured enterocytes or enteroids derived from wild-type mice (FIG. 5A) with BDNF (1 μg/ml) show reduced TLR4 signaling as manifest by reduced TLR4-induced cytokine expression after treatment with the TLR4 ligand LPS (50 μg/ml). Second, injection of BDNF (1 μg/kg) into wild type mice significantly reduced TLR4-mediated expression of the pro-inflammatory cytokines (FIG. 5B) in the intestinal mucosa 6h after LPS injection; protection was seen in ileum from NEC patients (IRB 00094036) treated with BDNF (FIG. 5C). Third, oral administration of BDNF (1 μg/kg) on each day of the model significantly protected against NEC development (FIGS. 5D-E), as revealed by improved histological integrity of the mucosa and reduced inflammatory cytokines. Fourth, the BDNF +/− mutant mouse (Jackson labs strain B6.129S4-Bdnf) showed significantly worse NEC as compared with wild type counterparts (FIGS. 5F-G) illustrating the importance of endogenous BDNF in restraining TLR4. Fifth, wild-type mice with NEC show reduced levels of BDNF in the intestinal mucosa (FIG. 5H-I), consistent with the reduction in glia. Sixth, mice lacking TrkB in the intestinal epithelium show severe NEC that is not protected by BDNF administration (FIG. 6). Most importantly, seventh, the administration of BDNF (1 μg/kg) significantly reversed the severe NEC observed above in the glial-KO mouse model (FIGS. 5H-K).

### Example 4

Identification and evaluation of novel agents that enhance enteric glial activity and reduce NEC.

The inventors next embarked on a strategy to identify novel therapeutic agents for NEC based upon their ability to enhance glial function and thus reduce TLR4 signaling and NEC. To do so, several libraries of FDA approved compounds were screened against the rat enteric glia cell line EGC/PK060399 (ATCC® CRL-2690™) for their ability to induce BDNF release, then the hits were validated by their ability to reduce NEC in mice. The lead hit, shown in FIG. 6A, is a C₁₃H₁₁NO₅ compound with MW 261.23Da, herein named “J11”. J11 induced BDNF release enteric glia in cultured enteric glia (FIG. 6B) and in the intestine after oral administration (20 mg/kg) in wild type mice (FIG. 6C). Importantly, J11 inhibited LPS (5 mg/kg)-mediated TLR4 signaling in the intestine of wild type mice (FIG. 6D) and significantly reduced NEC severity when administered orally (20 mg/kg/day) to 7d old mice (FIG. 6E). Strikingly, J11 (10 μM) significantly induced BDNF release and reduced TLR4 signaling (LPS 25 μg/ml) in premature human intestine obtained after surgery for NEC under IRB 00094036 (FIG. 6E), suggesting a potentially physiologically relevant role for this new compound.

Based upon the above studies, the inventors then tested their overriding hypothesis that the enteric glial play a critical, but previously unrecognized, role in restraining the exaggerated signaling of TLR4 in the intestinal epithelium thus preventing NEC. Without be held to any particular theory, the inventors believe that the protective effect of neonatal enteric glia occurs through the release of brain derived neurotrophic factor (i.e., BDNF), which inhibits intestinal epithelial TLR4 via the TrkB pathway. It is further thought that the paucity of enteric glia in the premature gut, in combination with TLR4-induced enteric glial loss, leads to exaggerated epithelial TLR4 signaling and NEC. Finally, the inventors now show that a recently discovered enteric glial activating agent termed “J11”, represents a novel strategy for NEC prevention and treatment.

### Example 5

Evaluation of how enteric glia-derived BDNF inhibits TLR4 signaling in the premature gut in the pathogenesis of NEC.

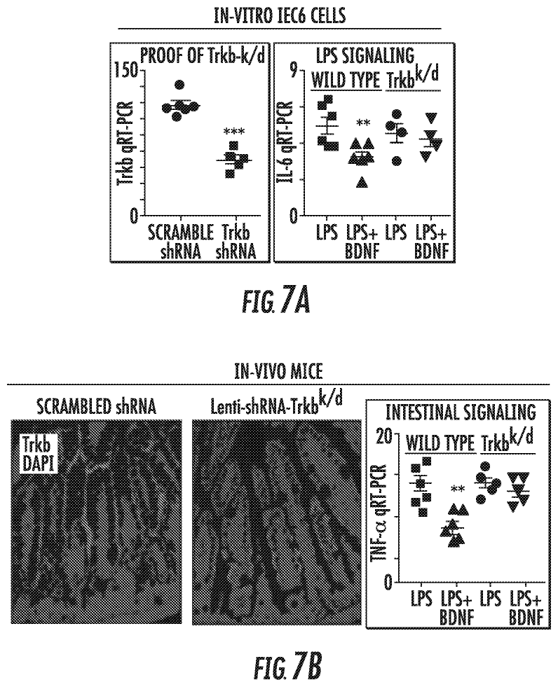

As shown in FIG. 5, the enteric glia inhibit TLR4 signaling in the intestinal epithelium via the release of BDNF, as glia KO mice, which have low BDNF levels, develop exaggerated TLR4 signaling and severe NEC which is reversed by the administration of BDNF. The inventors sought to determine how BDNF inhibits TLR4 and thus attenuates NEC. BDNF is known to signal principally via its receptor, namely tropomyosin-related kinase B (TrkB), which the inventors now show to be present in the intestinal epithelium (FIG. 7). The inventors have now knocked down TrkB in IEC-6 enterocytes (FIG. 7A) using lentiviral delivery of TrkB shRNA. The knockdown of TrkB prevented BDNF (1 μg/ml)-mediated protection of LPS (50 μg/ml) induced IL-6 expression in the IEC-6 cells (FIG. 7A). The inventors have also knocked down TrkB from newborn mouse intestinal epithelium by oral gavage of TrkB shRNA, (FIG. 7B), and as expected, mice deficient in TrkB in the intestinal epithelium show no protection of LPS-mediated TNF induction by BDNF (1 μg/kg, FIG. 7B), thus linking TrkB to BDNF signaling in the neonatal gut.

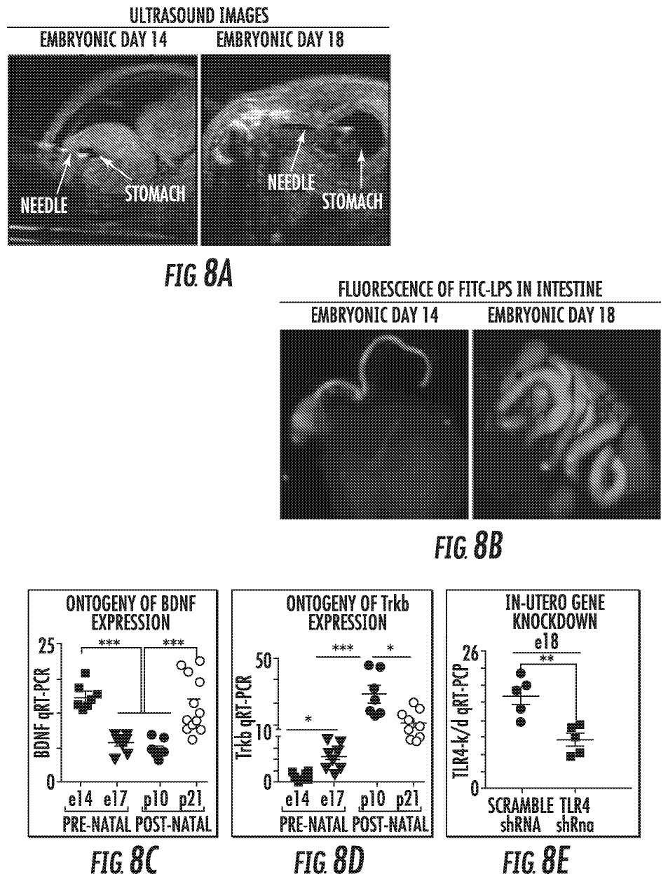

To uncover the mechanisms by which enteric glia can limit TLR4 signaling in the premature host, the inventors focused on the BDNF-TrkB pathway in the premature gut, when the host is at greatest risk for NEC development. To effectively interrogate how BDNF inhibits TLR4 signaling in the premature gut, the inventors turned to the in utero setting, in which the developing intestine, which has few glia (FIG. 3) also expresses high TLR4 (FIG. 2). The inventors have now determined that the premature bowel has low BDNF and high TrkB expression (FIG. 8).

In order to determine the effects of BDNF on TLR4, the inventors will first deliver BDNF (1-5 μg/embryo in max volume 5 μl/embryo) with or without the TLR4 agonist LPS (5-10 μg/embryo) directly into the lumen of the developing gut using our highly innovative system of backscatter microinjection. This system is shown in FIG. 8, as the inventors have published previously, and involves a laparotomy to the mother at day e16.5, followed by backscatter guided microinjection into the bowel. In each case, 4 pups are injected with ligand, and 4 with saline. The pups are routinely delivered to term, with an 80% survival. Intestine is harvested after euthanasia on days 1-5 to assess the degree of LPS-mediated TLR4 induction as in FIG. 7 and enterocyte apoptosis by TUNEL, important steps that lead to NEC.

Two parallel approaches were undertaken to assess whether BDNF is acting through TrkB as opposed to some other receptor. First, the inventors knocked down TrkB in the intestinal epithelium of the fetus in utero by injecting TrkB shRNA or TLR4 shRNA (see FIG. 8 for evidence of knockdown of TLR4). Second, the inventors injected the NTrk2 tmlDdg/J mutant mouse strain (David Ginty, Harvard, provided to Jackson labs) with the small molecule TrkB inhibitor 1NMPP1 (25 μM) which selectively inhibits TrkB in the mutant mice only, providing great selectivity. These mice are in the lab and are breeding well, and have preliminarily achieved TrkB inhibition. It is anticipated that administration of BDNF will decrease LPS-TLR4 signaling in the premature gut which will be lost in mice lacking TrkB.

Next the inventors determined in greater detail the signaling mechanisms by which BDNF-TrkB signaling inhibits TLR4. In the premature intestinal epithelium as in other cells, the inventors have shown that TLR4 activation requires the adaptor protein MyD88 which leads to NFkB induction and the release of pro-inflammatory cytokines, and which can be inhibited by the endogenous inhibitor IRAK-m. The inventors will thus investigate the roles of MyD88 or IRAK-m in mediating the mechanisms by which BDNF inhibits TLR4 signaling. The inventors will study MyD88 and IRAK-m expression, along with the expression of TLR4, in the fetal gut as above, and in cultured enteroids obtained from the bowel from the fetal to postnatal days e16.5-p5. IEC-6 cells or enteroids from wild-type and glial KO mice (as controls) will be treated with LPS (25-50 μg/ml corresponding to the doses used in our earlier studies) and BDNF (1-5 μg/ml), and after 1, 6 and 12h determine by RT-PCR and SDS-PAGE whether BDNF administration reduces expression of TLR4 itself (suggesting mRNA regulation or protein stability respectively) or MyD88 (suggesting downstream effects), or upregulates the known TLR4 inhibitor IRAK-m. Based upon these in vitro studies, the inventors will next perform validation studies in vivo, turning again to our in utero injection system in which we will deliver BDNF (1-5 pg/embryo) in the developing gut, or at postnatal days pl through p18, and assess the expression of TLR4, MyD88 and IRAK-m, as described above. In important controls, the inventors will perform studies in the TLR4 KO, MyD88 KO and TrkB knockdown mice, which are available in our lab, and which should not show any effect from BDNF administration.

### Example 6

Determination of the physiological significance of the above experiments by confirming whether the earlier reconstitution of the protective role of enteric glia (using BDNF) leads to protection from the subsequent development of NEC.

To do so, wild-type mice will be injected with BDNF (1-5 μg/embryo) in utero on days e16-19, and mice will be subjected to the model of NEC as in FIG. 1. Control mice will be either saline-injected or undergo TrkB knockdown 24h prior to BDNF injection. NEC will be induced on day 7 and severity assessed as in FIG. 1, and MyD88, IRAK-m and TLR4 expression will be measured by RT-PCR. In controls, the Nrtk2tm1Ddg mutant mice will be studied after injection with 1NMPP1 (1μg/kg) to block TrkB, in which BDNF should have no effect, and NEC should be severe. It is anticipated that the earlier BDNF administration will lead to stable protection from NEC by curtailing TLR4 signaling, through the pathways outlined above.

Given that BDNF would be predicted to cross the placental barrier, it will be assessed whether BDNF can be administered to the pregnant mother, as a means to dampen the degree of TLR4 signaling in the infant, and thus reduce the risk of NEC development in the pup. To do so, BDNF (1-5 μg/kg) will be administered to the pregnant mouse by i.p. on days post coitum (pc) 15.5 to 18.5, and then subject the pups to NEC, while control mothers will receive saline. The pups will then be assessed whether are protected as in FIG. 3. It is anticipated that these strategies which complement the enteric glia function in the premature pup/infant can be utilized to reduce NEC.

### Example 7

Enteric glia can be increased in the premature intestine through early colonization with probiotic bacteria to prevent NEC.

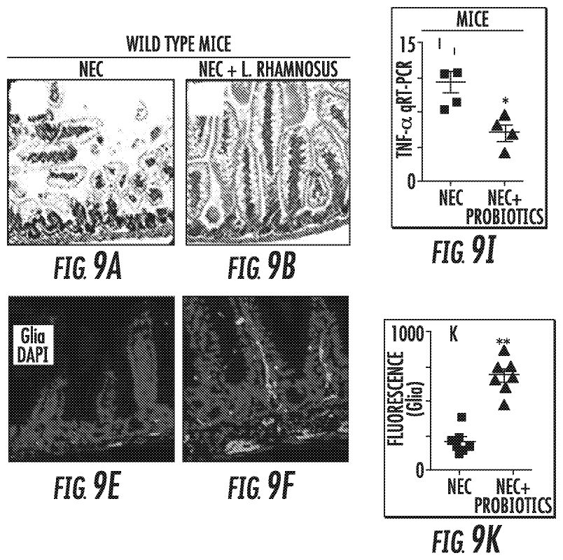

The enteric glia are highly dynamic structures which can be recruited into the lamina propria from a precursor pool in the muscularis propria in response to luminal bacteria. The ability to administer specific bacteria to the intestine provides an opportunity to potentially enhance enteric glia recruitment into the mucosa by altering the luminal bacteria. The inventors and others have shown that the administration of probiotic bacteria can attenuate NEC severity in mice and piglets, which the inventors and others have attributed in part to a reduction in TLR4 signaling. These findings suggest that probiotics—which have entered clinical use for NEC prevention despite an incomplete understanding of how they work—may act through increasing the enteric glia in the lamina propria, where they could inhibit TLR4. In support of this, the inventors now show that the administration of the probiotic Lactobacillus rhamnosus significantly increased the number of enteric glia in the intestinal mucosa (FIG. 9). The inventors will now test whether the oral administration of probiotic bacteria to the newborn mice can increase the number of enteric glia in the premature small intestine and thus reduce TLR4 signaling and attenuate NEC.

These studies will be performed in newborn mice on day 7, which approximates the human 28 week premature infant (i.e. closed eyes, no hair, elevated TLR4, reduced glia and reduced BDNF). Wild-type mice will be induced to develop NEC according to the model in FIG. 1 in the presence of either saline or probiotic bacteria (strain Lactobacillus rhamnosus, ATCC), and the degree of NEC severity will be assessed as above. It is expected that the administration of probiotic bacteria will lead to a reduction in NEC severity, consistent with our prior observations. Next, it will be determined whether the administration of probiotic bacteria (L. rhamnosus, but also the commonly used probiotic namely Bifidobacterium, both at 1×10⁵ CFU/ml) leads to an increase in enteric glia, by immunostaining for GFAP. It is expected to see an increase in enteric glia, consistent with the preliminary data in FIG. 9, heat killed bacteria will serve as controls. Therefore, we will next determine whether an induction in enteric glial cells by probiotic bacteria is required for the protection against NEC, and will repeat the above studies (i.e. the oral probiotics, saline, or heat-killed bacteria will be administered) in the glial KO mice strains in FIG. 3 (i.e. GFAP KO and PLP-1 KO mice). It is anticipated that the lack of glia will reverse the protective effect of probiotics, demonstrating the novel finding that the induction of enteric glia are required for the protection against NEC achieved by probiotics.

### Example 8

The inventors have previously shown that probiotic bacterial DNA is capable of achieving the protective effects of probiotic bacteria in mice and piglets, which acts via its receptor Toll like receptor 9 (TLR9). It will then be determined whether TLR9 activation is linked to the probiotic-induced recruitment of enteric glia to the lamina propria seen in FIG. 9, by repeating the above studies in the TLR9 KO mouse(breeding in the Hackam lab), and the gut-specific TLR9 KO system described in our recent publication. In control experiments, we will treat mice with non-probiotic DNA (i.e. obtained from E. coli), and will use the specific TLR9 agonist CpG-DNA (10 μg/ml) as a positive control. NEC will be induced, followed by assessment of severity and determine enteric glia density as in FIG. 4. It is anticipated that probiotic administration will not lead to enteric glia accumulation in the TLR9 knockout mouse.

### Example 9

Investigation of potential mechanisms by which TLR9 activation by probiotics leads to the recruitment of enteric glia in the protection against NEC.

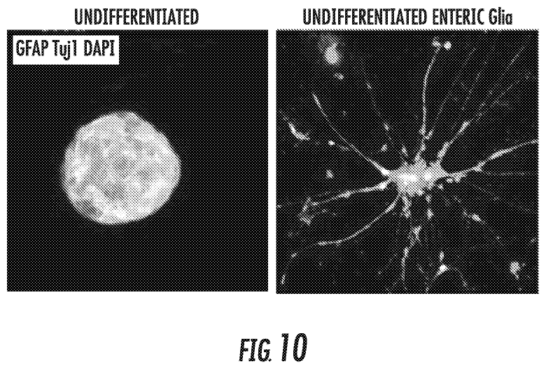

Previous authors have shown that enteric glia recruitment requires the transcription factor Sox10, raising the possibility that TLR9 signaling could induce Sox10 expression in mediating enteric glia recruitment. To evaluate this possibility, enteric glia precursor cells (neurospheres) will be isolated according to the methodology of Steinkamp et al. These precursor cell isolates have the potential to differentiate into glia or neurons, depending on the media used. As shown in FIG. 10, the inventors have experience with this technique, and show a sample neurosphere and a differentiated enteric glial cell from wild type mice. Mice will be treated with probiotics and probiotic DNA in vitro, and assess Sox10 expression. In parallel, neurospheres will be treated with non-probiotic DNA. It is expected that the probiotic DNA will lead to increased Sox10 expression and an enteric glia phenotype as in FIG. 10. To assess the physiological relevance of these findings, the inventors have generated tamoxifen and diphtheria toxin-inducible Sox10 KO mice by breeding ROSA26iDTR with Sox10-cre/ERT2; mice are fertile and viable but show hindlimb paralysis, confirming the Sox10 deletion genotype. The doses of tamoxifen/diphtheria toxin will be carefully titrated to regulate Sox10-DT inducible glia loss, and to minimize hindlimb paralysis. Wild type littermates will receive tamoxifen as a control, which in our experience causes no phenotype. Neurospheres will be isolated from these mice, which should not reveal enteric glia differentiation, and confirm these findings by administering probiotics to wild-type and Sox10-deficient mice and induce NEC. It is anticipated that the Sox10-glia-KO mice will show higher NEC severity which will not be reduced by probiotics. Taken together these findings will shed light on the pathways by which probiotics prevent NEC through recruitment of enteric glia.

### Example 10

Evaluation of the role of apoptosis in TLR4-induced enteric glia loss in NEC pathogenesis.

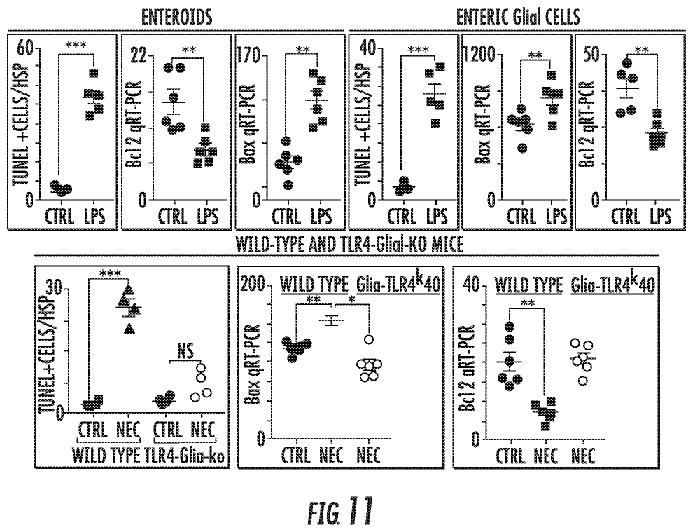

The inventors have shown that treatment of enterocytes (IEC-6 cells or enteroids) and enteric glia with the TLR4 ligand LPS (50 μg/ml for enterocytes, 1μg/ml for glia) leads to significant apoptosis of enterocytes or enteric glia in vitro, as revealed by expression of TUNEL (FIG. 11). Further, NEC in wild type mice leads to enteric glial apoptosis in vivo that is not seen in TLR4-glia-CKO mice (FIG. 11). In seeking the mechanisms involved, it was noted that apoptosis is regulated in part by the balance of the pro-apoptosis gene Bax and anti-apoptosis Bcl2. The inventors now show that the pro-apoptosis molecule Bax is increased in enterocytes and enteric glia after LPS treatment and in the lamina propria of wild-type but not TLR4-glial-CKO mice with NEC, showing that TLR4-induced Bax could mediate enteric glia apoptosis (FIG. 11). To test this directly, Bax will be deleted from the enteric glia, by breeding our GFAP- and PLP-1-cre strains with the Bax-loxp mouse from Jax. Bax deletion will be confirmed by RT-PCR and immunostaining, and these mice subjected to NEC. The initial pups appear to show Bax deletion. It is anticipated that these mice will show reduced NEC severity and no loss of enteric glia as compared with wild type mice. In parallel, we will assess the role of the anti-apoptotic gene Bcl2, which we show to be reduced in enterocytes and enteric glia after LPS, and in wild type but not TLR4-glia-CKO mice with NEC (FIG. 11). Bcl2 will be deleted from the enteric glia by breeding the Bcl2-loxp mice from Jax with our GFAP- and PLP-1-cre mice and confirm its loss by RT-PCR and immunohistochemistry. NEC will then be induced in these mice and increased glial loss and increased NEC severity is expected compared with wild type strains, thus supporting the importance of enteric glia apoptosis in the pathogenesis of NEC.

### Example 11

Pharmacological inhibition of enteric glia apoptosis as a strategy to reduce NEC severity.

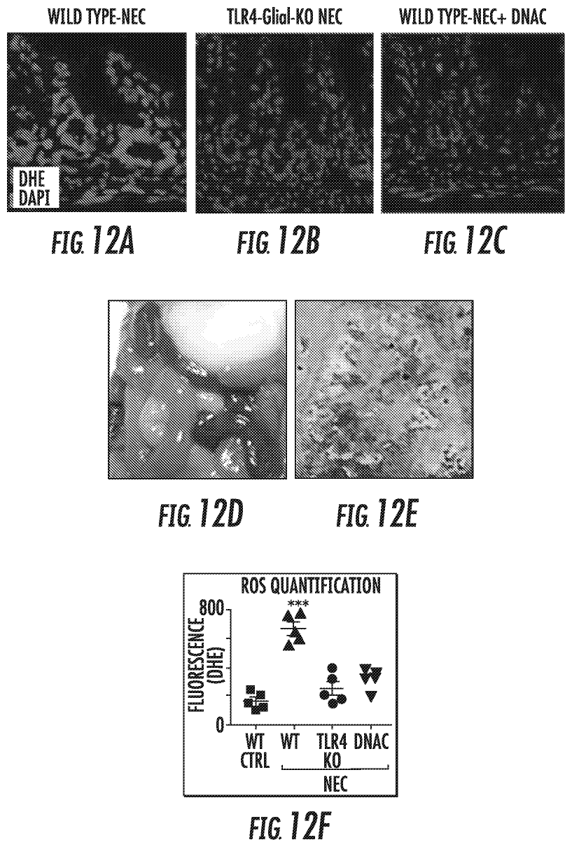

It is noted that the release of reactive oxygen species, as occurs in the lamina propria after TLR4 signaling in NEC (FIG. 12), is a powerful inducer of apoptosis in enteric glia. Wild-type mice show increased ROS in the lamina propria as revealed by the marker dihydroethidium (DHE) that is not seen in the glia-TLR4 KO mice (FIG. 12), suggesting a role for ROS in enteric glial apoptosis. To test this possibility directly, a novel dendrimer-drug conjugate nanoparticle, D-NAC, will be administered, which has been shown, when administered orally, to accumulate in the lamina propria and quenche the ROS production, as indicated in FIG. 12. The efficacy of D-NAC in the model of experimental NEC at a drug dose of 10 mg/kg, showed excellent incorporation into the lamina propria (FIG. 12). D-NAC will be administered (p.o., 10 mg/kg/), NEC will then be induced, and enteric glia and enterocyte apoptosis will be quantified as in FIG. 11. In control experiments, mice will be treated with NAC alone, which should block ROS in all cells, and unconjugated dendrimer, which should have no effect. TLR4-glia-CKO mice will serve as an additional control, in which administration of D-NAC should offer no additional protection. It is thought that the administration of D-NAC will reduce enteric glia loss and attenuate NEC.

### Example 12

Evaluation of the role of TLR4 antagonists in preventing enteric glia loss and reducing NEC.

Having shown that mice lacking TLR4 on the enteric glia are protected from NEC (FIG. 3), the inventors will determine whether pharmacologic inhibition of TLR4 on the enteric glia can prevent enteric glia apoptosis and NEC development. To do so, the inventors will use a recently discovered a novel family of TLR4 inhibitors, the lead compound of which is a tetra-acetylated aminoglycoside of molecular weight 389 (called “C34”), which inhibits TLR4 binding by blocking the LPS binding pocket on TLR4, and which prevents NEC when administered orally to both mice and piglets. A series of novel and highly potent analogs to C34 (FIG. 13) were recently synthesized, which are capable of inhibition of TLR4 in vitro, i.e., compounds 1-8. C34 (compound 1) or its analogs will be evaluated for whether they can inhibit TLR4 more selectively in the enteric glia, and thus prevent glial apoptosis and NEC. The ability of the C34 analogs to inhibit TLR4-mediated apoptosis in the enteric glia versus enterocytes in vitro, will be measured by treating enteroids or IEC-6 cells with LPS (25 μg/ml) in combination with analogs (those in FIG. 13 plus others that we have synthesized) at increasing concentrations (from 1-50 μg/ml), and assess apoptosis of enterocytes and glia as in FIG. 11. Enteroid cultures will be performed. Those analogs with the ability to prevent apoptosis preferentially in the enteric glia will be selected. Using this screen, saline will be administered (as a control), or the analogs, at doses from 5 to 10 mg/kg by mouth, to wild-type mice in the model of NEC, and then assess enteric glia apoptosis and NEC severity as in FIGS. 3 and 11.

In control experiments, the compounds will be administered to TLR4-glial-CKO mice to assess off target effects. It is anticipated that we will see TLR4-inhibitors with predilection for enteric glia, and thus a novel approach to treating NEC, without potential immune consequences of enterocyte TLR4 inhibition.

### Example 13

Identification of other novel small molecule agents that can enhance enteric glia with high specificity and efficacy, to develop novel approaches for the prevention and treatment of NEC.

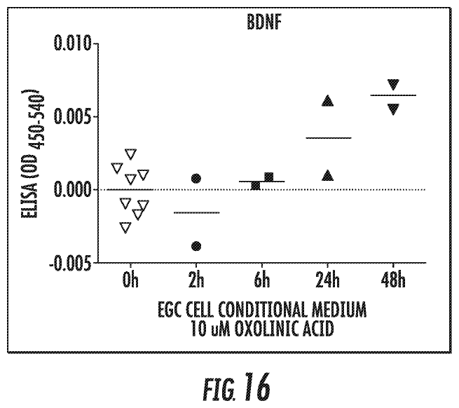

A dose-response curve was established for oxolinic acid (J11) in inducing BDNF release in the enteric glia cell line EGC/PK060399, and will be used evaluate a safety profile based upon its mean effective dose in mice. As shown in FIG. 16, enteric glial cells (EGC/PK060399egfr, ATCC® CRL-2690™) were treated with 10 μM J11, and the conditional medium were harvested at 2, 6, 24 and 48 hours after treatment. The BDNF secretion was quantified using BDNF ELISA kit following the manufacturer's protocol. The amount of BDNF was time dependent with the greatest secretion after 48 hours of exposure.

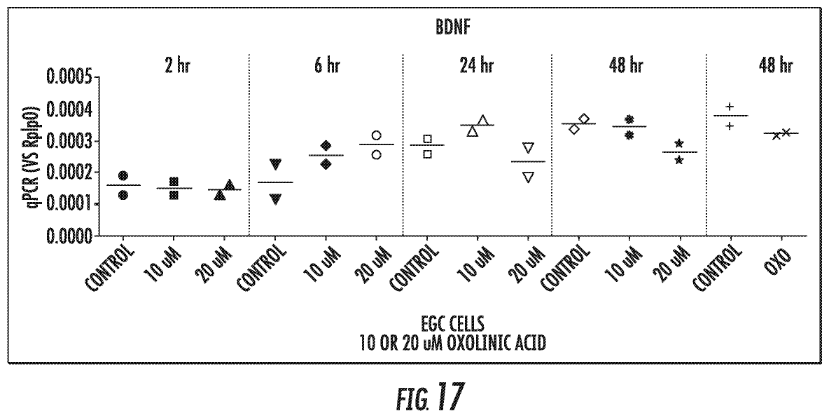

As seen in FIG. 17, BDNF mRNA expression by enteric glial cells was stimulated with 10 μM and 20 μM J11, with maximal expression after 24 hours. The enteric glial cells (EGC/PK060399egfr, ATCC® CRL-2690™) were treated with 10 μM and 20 μM J11, and the enteric glial cells were harvested at 2, 6, 24 and 48 hours after treatment. Total RNA was isolated using the RNeasy mini kit following the manufacturer's protocol. Complementary DNA was synthesized from 0.5 μg RNA using M-MLV reverse transcriptase. The qPCR analysis was performed with the Bio-Rad CFX96 Real-Time System (Biorad, Hercules, Calif.). The relative BDNF mRNA expression was normalized against the expression of housekeeping gene RPLO.

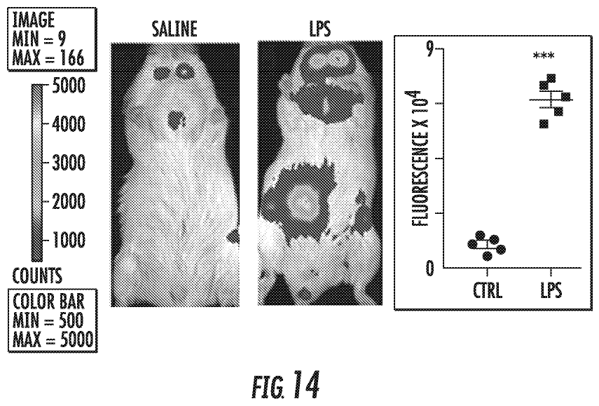

In parallel, the functional relevance via reduced LPS-induced TLR4 signaling in the intestine will be confirmed, which we will assess in vivo by injecting newborn transgenic mice that express NFkB on the luciferase promoter (NFkB-luc mice) with LPS (1 mg/kg) along with luciferin, such that luciferase emission provides a readout of TLR4-NFkB signaling (see FIG. 14). Escalating doses of J11 (from 1 to 100 mg/kg) will be tested, and BDNF release measured as a marker of enteric glia cell activation by ELISA.

To establish a safety profile for J11 in mice, escalating doses of J11 will be used around the mean effective dose identified above, and obtain tissue for evaluation of standard cardiac, neuronal, hematologic and renal parameters in wild-type mice, as well as enteric glial KO mice (GFAP KO and PLP-1 KO mice) to assess off-target effects. This dose is then used to establish the mean effective dose of J11 in preventing experimental NEC in mice by administration of J11 at 6 h, ld, or 2d prior to the onset of NEC, and then assess TLR4 expression, BDNF release, and LPS induced IL-6 upregulation in the gut NEC severity as in FIG. 3. To evaluate whether the effects of J11 on reducing NEC severity occur via reduction of TLR4 signaling, the studies in our TLR4-^{villin-over} mice will be repeated which over-express TLR4 on the intestinal epithelium on a TLR4^{-/-} background and assess whether we lose the degree of protection of J11. Having shown that administration of J11 can prevent NEC (FIG. 7), we will next investigate whether J11 can treat established NEC jn mice. To do so, wild-type mice will be subjected to NEC for 1, 2 or 3 days, give J11 at the dose determined above for an additional 1, 2 or 3 days, and NEC severity assessed along with TLR4 signaling within the gut. It is expected that these studies will show that J11 can both prevent and treat experimental NEC, and to determine the appropriate dose required.

### Example 14

Design, synthesis and determination of structure-activity relationships (SAR) of novel analogs of J11.

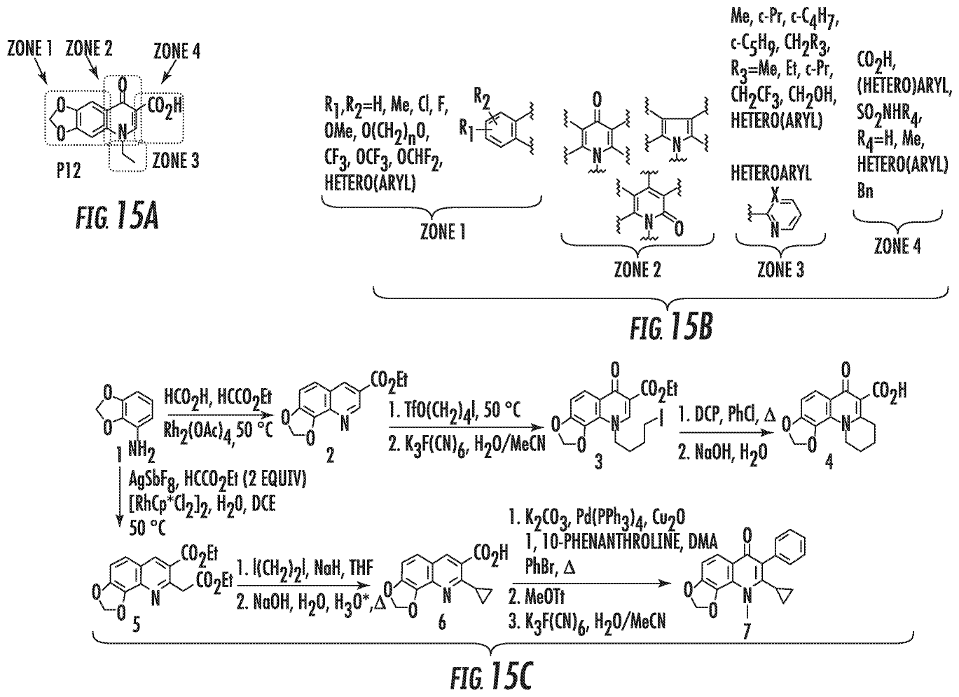

The inventors seek to synthesize analogs of J11, a process that is expected to reveal more potent and/or selective derivatives through rational modifications of its primary structure. Chemical modifications of J11 will provide opportunities to remove off-target effects that could narrow the safety profile of this treatment. Based upon its chemical structure (FIG. 6), but without being held to any particular theory, J11 is expected to inhibit DNA gyrase and thus could possess antibiotic properties. The process will counter-screen for antibiotic effects by performing serial antibiotic assays using stool from mice and humans with NEC, on agar plates. Those compounds will be selected that have maximum enteric glial activating properties (i.e. BDNF release) and TLR4 reduction, with fewest potentially confounding antibiotic effects. The approach to the J11 analog preparations (FIG. 15) is to systematically modify four zones in the target structure (Panel A), introduce 3-10 modifications at each of the zones and substituents, and use substituents at the C-2 position to manage antibiotic properties, since it is well known that substitution at this position of 1-alkyl-1,4-dihydro-4-oxo-3-quinolinecarboxylic acids abolishes antibacterial activity. The goal is to generate 40-60 close structural variants of J11 and determine their properties in BDNF release- and NFkB luciferase screens followed by the studies in experimental NEC to determine their structure-activity profile. FIGS. 15B-C show the substitutions envisioned for each of the zones 1-4 that will be accompanied by target molecular modeling to prioritize designs. The metabolic profile of J11 and each analog will be benchmarked via liver microsome cytochrome P450 metabolism studies, and the major metabolite(s) will be determined by LC/HRMS (Liquid Chromatography High Resolution Mass Spectrometry). After demonstrating safety and efficacy within a range of doses (0.1-10 mg/kg), these novel analogs will then be used in experimental NEC in mice, and assess NEC severity, TLR4 expression, and BDNF release.

### Example 15

Determination of J11 in preventing or treating NEC in a piglet model and in human tissue ex vivo.

In order to provide a bridge to the potential clinical use of J11 or its analogs, the inventors have established a piglet model of NEC. The piglet is the approximate size of a human premature infant (1000-1200g), and its intestine expresses TLR4, and shares physiologic and structural properties with the premature human. The piglet model involves delivery of premature piglets via cesarean section at 92% gestation by gavaging a mix of formula feeds containing Pepdite Junior (Nutricia), MCT oil, and whey (at 20 ml/kg every 3 h (120 mL/kg/day) for 4 days which was supplemented with enteric bacteria from an infant with surgical NEC. J11 or its analogs will be administered with the infant formula at concentrations 5-20 mg/kg, determine NEC severity, and evaluate its role in preventing or treating NEC. For NEC prevention in piglets, J11 or its analogs will be administered for 24 or 48 h prior to NEC induction; for NEC treatment, J11 will be administered at 24, 48 or 72 h after NEC induction. In all cases, piglets will be orally gavaged once daily (20 mg/kg/day); saline will be administered as a control. TLR4-mediated cytokine induction in the intestine will be evaluated as in FIG. 3. In order to define whether a reduction in TLR4 signaling is required for the protection from NEC, LPS (5-10 mg/kg) or bacteria from a NEC infant (1×10⁵ cfu/mL×10 mL) will be administered to the piglets then induce NEC and determine whether TLR4 activation can reverse the protection of J11. Finally, we will test the analogs in human tissue ex vivo for the ability to reduce inflammation in freshly discarded tissue from NEC resections as in FIG. 6 (IRB 00094036). It is anticipated that novel J11 analogs with superior pharmacokinetic parameters and efficacy than the original compound, will be identified, providing an important bridge to potential clinical use of J11 or its analogs.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Use of an anti-oxidant and/or anti-inflammatory composition in an effective amount for the prevention or treatment of necrotizing enterocolitis (NEC) in the intestine of a pre-term or neonatal mammalian subject.

2. Use of a composition comprising dendrimer nanoparticles wherein the dendrimer nanoparticles comprise one or more ethylene diamine-core poly(amidoamine) (PAMAM) hydroxyl-terminated dendrimers covalently linked to at least one biologically active agent, in an amount effective to suppress or inhibit NEC in the intestine of a pre-term or neonatal mammalian subject.

3. Use of a glial agonist composition in an effective amount for the prevention or treatment of NEC in the intestine of a pre-term or neonatal mammalian subject.

4. Use of a TLR4 antagonist in an effective amount for the prevention or treatment of NEC in the intestine of a pre-term or neonatal mammalian subject.

5. The use of claim 3, wherein the method further comprises administration of an effective amount of Brain Derived Neurotrophic Factor (BDNF).

6. The use of claim 5, wherein the BDNF is administered to the subject at a dose range of 0.01 μg/kg to up to about 100 mg/kg.

7. The use of claim 6, wherein the BDNF is administered orally.

8. The use of either of claim 1 or 2, wherein the antioxidant composition is selected from the group consisting of: a-tocopherol, ascorbic acid, Mn(III)tetrakis (4-benzoic acid) porphyrin, a-lipoic acid, n-acetylcysteine, and n-acetylcysteine amide.

9. The use of either of claim 1 or 2, wherein the anti-inflammatory composition is selected from the group consisting of: methyl prednisone, dexamethasone, non-steroidal anti-inflammatory agents, including COX-2 inhibitors, corticosteroid anti-inflammatory agents, gold compound anti-inflammatory agents, immunosuppressive and anti-inflammatory agents, salicylate anti-inflammatory agents, ranibizumab, and minocycline.

10. The use of either of claim 8 or 9, wherein the antioxidant and/or anti-inflammatory composition is administered to the subject at a dose range of 0.01 μg/kg to up to about 100 mg/kg.

11. The use of claim 10, wherein the antioxidant and/or anti-inflammatory composition is administered orally.

12. The use of claim 3, wherein the glial agonist composition is selected from the group consisting of: methscopolamine bromide, ketoprofen, estradiol cypionate, anisodamine hydrobromide, docosanol, oxolinic acid (J11), xylose, benurestat, ioxilan, chlorazanil hydrochloride, OSI-420, laptinib ditosylate, erlotinib HCL, erlotinib, and gefitinib, with or without a pharmaceutically acceptable carrier.

13. The use of claim 12, wherein the glial agonist composition is administered to the subject at a dose range of 0.01 pg/kg to up to about 100 mg/kg.

14. The use of claim 13, wherein the glial agonist composition is administered orally.

15. The use of claim 4, wherein the TLR4 antagonist is selected from the group consisting of: compounds 1 to 8 as shown in FIG. 13.

16. The use of claim 15, wherein the TLR4 antagonist composition is administered to the subject at a dose range of 0.01 pg/kg to up to about 100 mg/kg.

17. The use of claim 16, wherein the glial agonist composition is administered orally.

18. The use of claim 1, wherein the method further comprises administration of an effective amount of a probiotic bacteria.

19. The use of claim 18, wherein the probiotic bacteria is Lactobacillus rhamnosus.

20. The use of claim 18, wherein the probiotic bacteria is Bifidobacterium.

21. The method of either of claim 19 or 20, wherein the probiotic bacteria is administered is between about 1×10⁴ to about 1×10⁶ CFU.

22. Use of a TLR4 antagonist having the following formula: 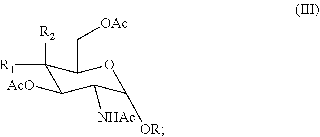
wherein R, R₁, and R₂ are each independently H or a straight chain or branched lower alkyl, lower alkenyl, lower alkynyl, alkoxy, cycloalkyl, heterocycloalkyl, aryl, arylalkyl, heteroaryl, and heteroarylalkyl, or a pharmaceutically acceptable salt, solvate, stereoisomer, or derivative thereof, in the uses of any of claims 3, 5, 12-14 and 17.

23. The use of claim 22, wherein the TLR4 antagonist of formula III, R is isopropyl or cyclohexyl, and R₁ and R₂ are independently H or acetoxy.

24. Use of a glial agonist having the following formula: 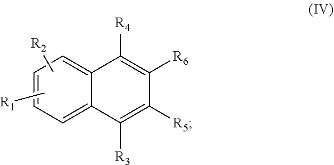
wherein R₁ and R₂ are independently H, C₁-C₆ alkyl, halo, alkoxy, alkyhalo, alkoxyhalo, aryl, heteroaryl, and when R₁ and R₂ are in combination, O(CH₂)ₙO, R₃ is H, C₁-C₆ alkyl, halo, alkoxy, alkyhalo, alkoxyhalo, aryl, heteroaryl, and substituted heteroaryl, R₄ and R₅ are alternatively H or O, R₆ is H, C₁-C₆ alkyl, halo, alkoxy, alkyhalo, alkoxyhalo, sulfonamide, alkysulfamido, aryl, heteroaryl or a pharmaceutically acceptable salt, solvate, stereoisomer, or derivative thereof, in the uses of any of claims 3, 5, 12-14 and 17.

25. The use of claim 24, wherein R₁ and R₂ are selected from the consisting of H, CH₃, Cl, F, OCH₃, CF₃, OCF₃, OCHF₂ and heteroaryl.

26. The use of claim 25, wherein R₃ is selected from the consisting of methyl, propyl, butenyl, pentenyl, and CH₂R₇, wherein R₇ is selected from the group consisting of methyl, ethyl, propyl, CH₂CF₃, CH₂OH, and heteroaryl.

27. The use of claim 25, wherein R₆ is CO₂H, heteroaryl, and SO₂NHRs wherein Rs is H, CH₃, heteroaryl, and phenyl.
